(19) United States
(12) Patent Application Publication
(10) Pub. No.: US 2021/0085415 A1
(43) Pub. Date: Mar. 25, 2021

(54) METHODS AND SYSTEMS FOR AN ACCESSORY HOLDER

(71) Applicant: GE Precision Healthcare LLC, Milwaukee, WI (US)

(72) Inventors: Ross Christopher Stalter, Hartland, WI (US); Robert Andrew Meurer, Waukesha, WI (US)

(21) Appl. No.: 16/579,690

(22) Filed: Sep. 23, 2019

(51) Int. Cl. A61B 50/20; A61B 8/00

(52) U.S. Cl. CPC A61B 50/20; A61B 8/4444; A61B 8/4477

(57) ABSTRACT

Various methods and systems are provided for an accessory holder. In one example, the accessory holder has a base plate with a front edge and a rear edge, and an inlet extending from the rear edge to an opening in the base plate. The opening has oppositely arranged, rebounding contact seats configured to bend away from the accessory inserted into the opening while exerting a pressure on the accessory.

### FIELD

Embodiments of the subject matter disclosed herein relate to a holder for a medical device, and more specifically, to a probe holder for an ultrasound system.

### BACKGROUND

Ultrasound imaging utilizes high-frequency sound waves to produce images of organs, tissues, or blood flow. The sound waves are produced by an ultrasound probe or transducer and transmitted in pulses. Reflection of the sound waves by boundaries between organs, tissues, bones, etc., are detected by the probe and relayed to a control unit where the reflected waves are converted to a two dimensional image.

In some instances, a compact, readily transportable ultrasound imaging system is desirable. By configuring the system to be portable, rapid evaluation of patient condition is provided, independent of location. Portable ultrasound imaging systems may expand point-to-care diagnostics and allow faster treatment protocols to be identified and applied to patients. As such, the ultrasound imaging system may be packaged to enable transport of all components of the system in unison. For example, the system may be adapted with structures to retain accessory devices, such as probes, secured to a main body of the system to minimize dislodgement of the accessory devices and separation of the devices from the main body.

### BRIEF DESCRIPTION

In one embodiment, an accessory holder comprises a base plate with a front edge and a rear edge, and an inlet extending from the rear edge of the base plate to an opening in the base plate, wherein the opening includes oppositely arranged, rebounding contact seats that partially close the opening, where the contact seats are configured to bend away from an accessory inserted into the opening while exerting a pressure on the accessory. In this way, the accessory may be secured to a portable system and readily decoupled from the accessory holder when operation of the portable system is demanded.

Furthermore, the accessory holder may be used for a variety of accessories. For example, medical devices, such as ultrasound probes, as well as non-medical devices, such as bar code scanners, of similar shape and size may be retained by the hand-held device holder. As such the accessory holder may be coupled to various types of systems.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows an example ultrasonic imaging system according to an embodiment of the disclosure.

FIG. 2 shows an example of an ultrasound probe that may be used in the ultrasonic imaging system.

FIG. 3 shows a first perspective view of an example of a holder for the ultrasound probe of FIG. 2.

FIG. 4 shows a second perspective view of the holder of FIG. 3.

FIG. 5 shows a third perspective view of the holder of FIGS. 3-4.

FIG. 6 shows a front view of a first portion of the holder of FIGS. 3-5.

FIG. 7 shows a front view of a second portion of the holder of FIGS. 3-6.

FIG. 8 shows the holder of FIGS. 3-7 implemented in a portable ultrasound imaging system according to an embodiment of the disclosure.

FIG. 9 shows an example of an opening of the holder of FIGS. 3-8 in a first configuration.

FIG. 10 shows an example of the opening of the holder of FIGS. 3-9 in a second configuration.

FIG. 11 shows an example of a set of dimpled contact seats of a holder for an ultrasound transducer according to an embodiment of the disclosure.

### DETAILED DESCRIPTION

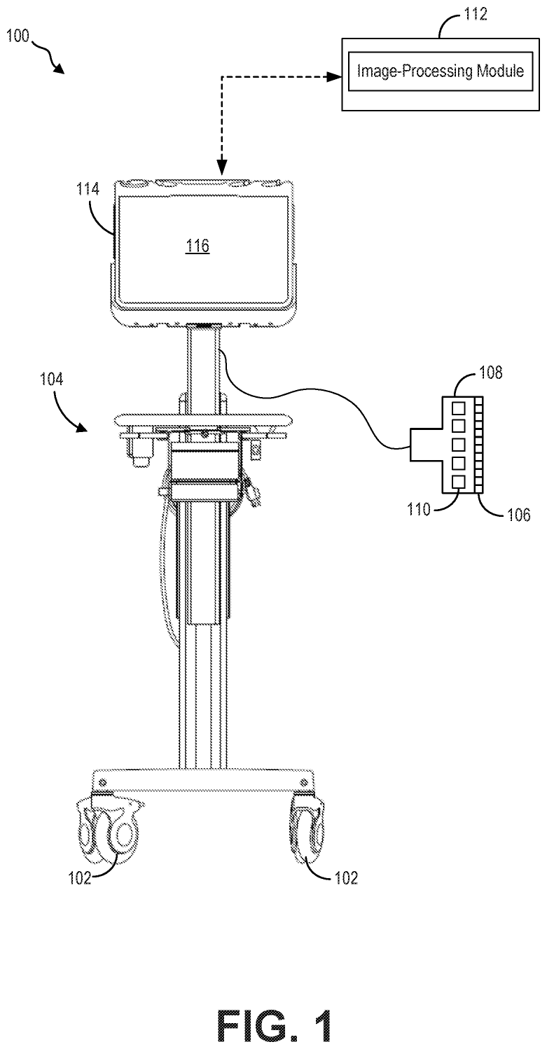

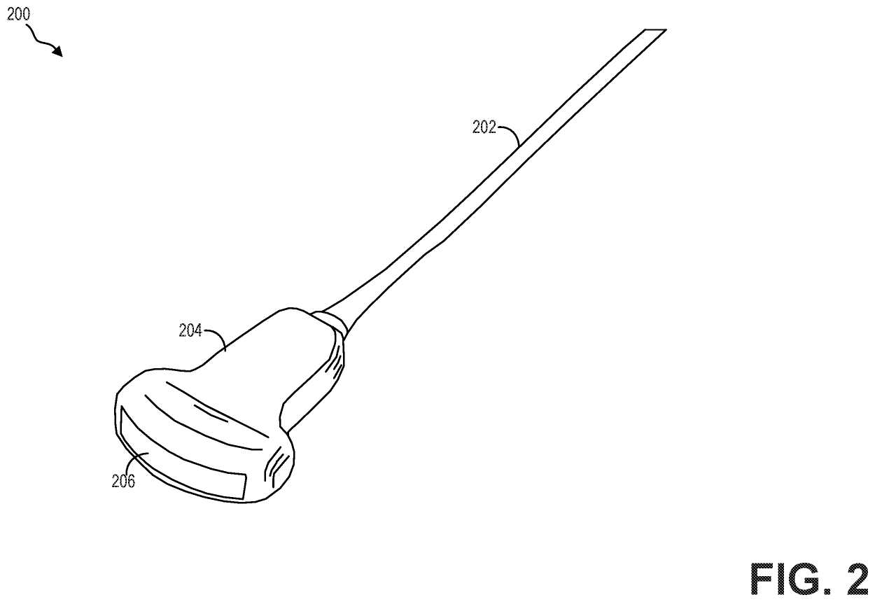

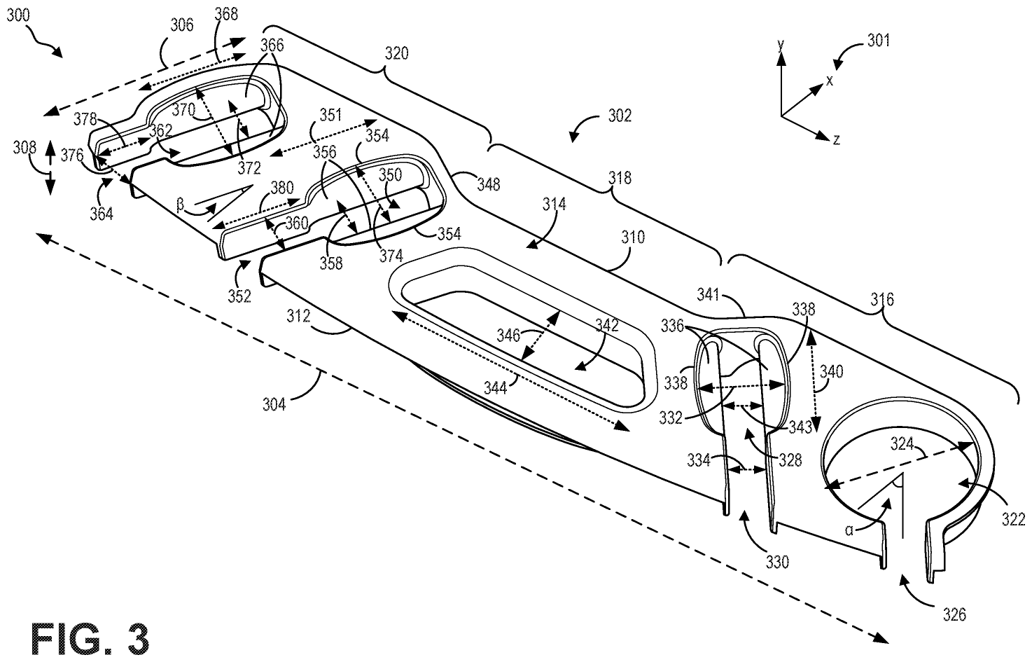

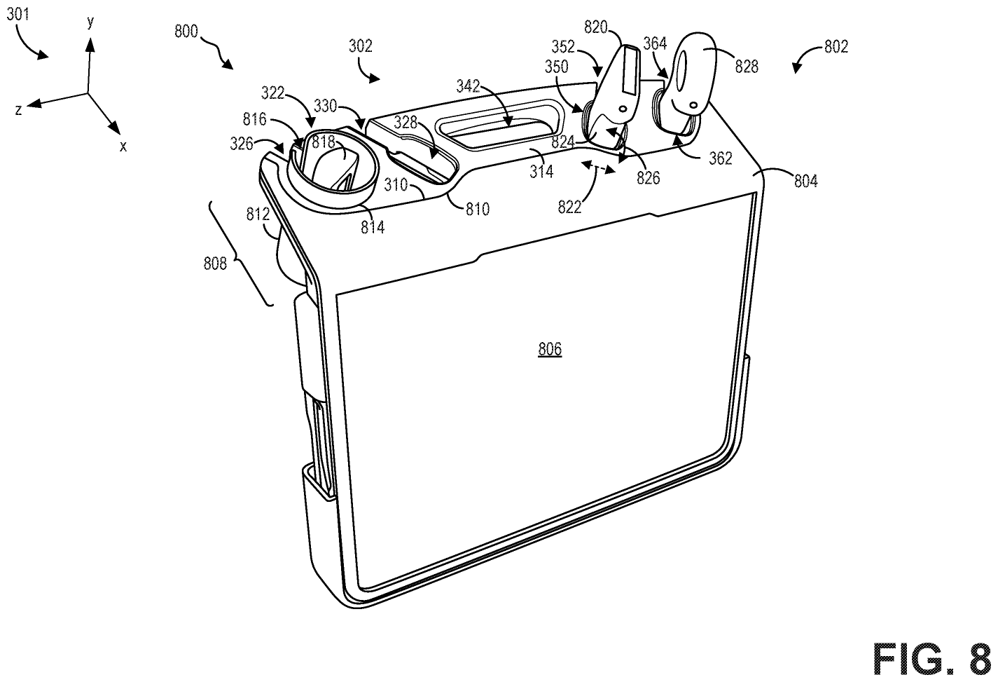

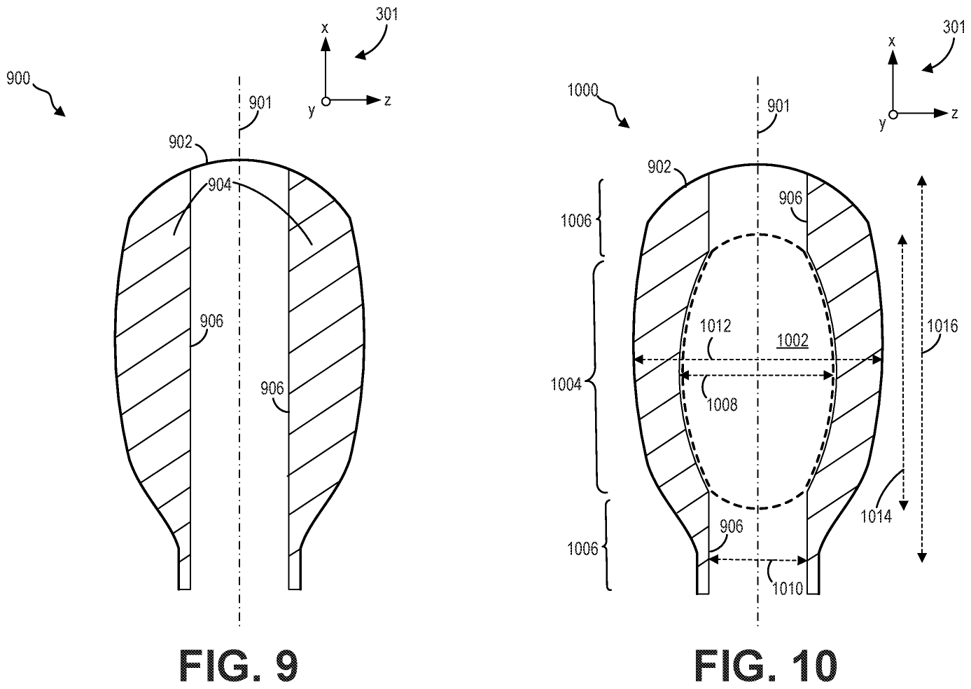

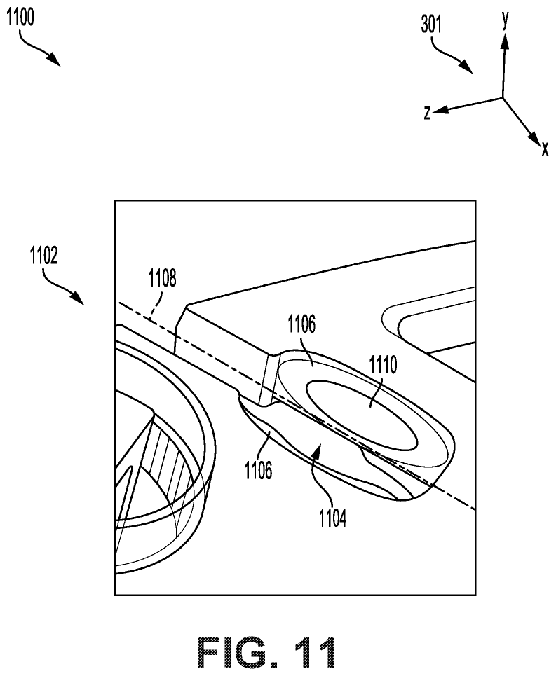

The following description relates to various embodiments of an accessory holder. In one example, the accessory holder may be used in an ultrasound imaging system, such as the ultrasound imaging system shown in FIG. 1, to support at least one ultrasound transducer or probe, such as the ultrasound probe shown in FIG. 2. The probe may be secured to the ultrasound imaging system by coupling the accessory holder to the ultrasound imaging system, providing a flexible structure for maintaining a position of the probe adjacent to the ultrasound imaging system. The accessory holder is depicted from different views in FIGS. 3-7. In FIG. 8, the accessory holder is shown implemented in the ultrasound imaging system. An opening of the accessory holder is shown in detail in FIGS. 9 and 10. The opening may be adapted with flexible contact seats that may be in a first configuration illustrated in FIG. 9 when the opening is not engaged with the probe, and in a second configuration shown in FIG. 10 when the probe is inserted into the opening. In some examples, the flexible contact seats may be adapted with indentations, or dimples, to increase a flexibility of the contact seats, as illustrated in FIG. 11.

FIGS. 1-11 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

An ultrasound imaging system is an example of an assembly that may utilize an accessory holder to secure a positioning of one more accessories coupled to the assembly. Ultrasound imaging is a widely used method for providing visual imagery of tissues, organs, and blood flow of a subject. Conventionally, an ultrasound imaging system may be located at a dedicated site, such as a clinic or hospitable, requiring patients to travel to the site to obtain a medical diagnosis. However, portable ultrasound imaging systems, such as hand-held or strap-carried units, allow the diagnostic capabilities of the ultrasound imaging to instead be provided at a location of the patient. For example, during an athletic event, if an athlete sustains hard impact and cannot continue activity, a medical practitioner may carry a portable system to the athlete and quickly evaluate the athlete's condition based on results of an ultrasound image. Suitable treatment may be immediately applied. As such, the portable systems may be self-supporting units that are easily transported to a treatment site.

An ultrasound imaging system includes accessory devices coupled to a main body of the system via cables. For example, the ultrasound imaging system may include at least one transducer probe attached to the main body by at least one cable. The probe enables scanning of a patient to obtain an ultrasound image and thus is maintained proximate to the main body to be easily accessed when scanning is to be performed. During transport of a portable ultrasound system, the system may experience vigorous bouncing and jostling, for example, if the system is carried while running. As the probe may be subject to degradation upon contact with objects or surfaces, securing of the probe to the main body of the portable ultrasound system is desirable.

In one example, one or more accessory devices of an ultrasound imaging system may be coupled to the ultrasound imaging system by a holder that may be removably or unremovably attached to a main body of the system. The holder may include a base plate adapted with openings shaped to receive an accessory device such as a probe. The openings may include a set of flexible, rebounding, bubble-like structures, arranged on opposing inner surfaces of each of the openings. A distance between the set of structures, or contact seats, within an opening may be configured to be smaller than a width of the probe which allows the set of contacts seats to exert pressure on the probe when the probe is inserted between the set of contact seats. The pressure retains a position of the probe within the holder, even when the ultrasound imaging system is transported.

The ultrasound imaging system may be sterilized between each usage. In order to minimize an amount of time spent cleaning the ultrasound imaging system the holder may be shaped to reduce sharp corners and/or crevices that may be difficult to clean thoroughly with a sterilizing wipe. An example of a holder for a medical diagnostic system, such as an ultrasound imaging system, is provided below, with reference to FIGS. 3-10, following a general description of the ultrasound imaging system and ultrasound probe.

It will be appreciated that while the accessory holder described herein is shown coupled to an ultrasound system, e.g., as shown in FIG. 8, the ultrasound system is a non-limiting examples of a system in which the accessory holder may be implemented. In other words, the accessory holder may be utilized in numerous other medical systems as well as non-medical systems. Any accessory having a similar geometry and similar dimensions to an ultrasound probe may be similarly maintained in place by the accessory holder. The accessory may be readily adapted to attach to other types of systems including portable and non-portable systems.

Turning now to FIG. 1, an example of an ultrasound imaging system 100 is illustrated, according to one embodiment. As shown, the system 100 includes multiple components. The components may be coupled to one another to form a single structure, e.g., a portable unit as shown in FIG. 1, or may be separate but located within a common room, or may be remotely located with respect to one another. Optionally, the system 100 may be a unitary system that is capable of being moved (e.g., portably) from room to room. For example, the system 100 may include wheels 102, may be transported on a cart 104, or may be hand-held or carried by an attached strap (not shown).

In the illustrated embodiment, the system 100 also includes an array of elements 106, for example, piezoelectric crystals, within a diagnostic ultrasound probe 108 to emit pulsed ultrasonic signals into a body or volume (not shown) of a subject. Furthermore, the probe 108 is outfitted with one or more actuators 110 capable of receiving signals from a system controller 112, as described further below, in order to output tactile feedback to the user. The elements 106, the one or more actuators 110, and the probe 108 may have a variety of geometries.

The system controller (e.g., electronic controller) 112 of the system 100 includes a plurality of modules, which may be part of a single processing unit (e.g., processor) or distributed across multiple processing units. The system controller 112 may be integrated into a main body 114 of the system 100 or may be a separate unit. When integrated into the main body 114, the main body 114 may be adapted to be detached from the cart and transported to a remote location. The system controller 112 is configured to control operation of the system 100. For example, the system controller 112 may include an image-processing module that receives image data (e.g., ultrasound signals in the form of RF signal data or IQ data pairs) and processes image data. Acquired ultrasound information may be processed in real-time during an imaging session (or scanning session) as the echo signals are received.

The system controller 112 is operably connected to the main body 114 that enables an operator to control at least some of the operations of the system 100. The main body 114 may include hardware, firmware, software, or a combination thereof that enables a user (e.g., an operator) to directly or indirectly control operation of the system 100 and the various components thereof. As shown, the main body 114 includes a display area 116. In some embodiments, the main body 114 may also include one or more input devices, such as a physical keyboard, mouse, and/or touchpad (not shown). The display area 116 also communicates information from the system controller 112 to the operator by displaying the information to the operator. The display area 116 and/or the main body 114 may also communicate audibly. The display area 116 is configured to present information to the operator during the imaging session. The information presented may include ultrasound images, graphical elements, user-selectable elements, and other information (e.g., administrative information, personal information of the patient, and the like).

As described above, an ultrasound transducer or probe may include an array of elements configured to generate ultrasonic signals and actuators to receive reflected ultrasonic signals. An exemplary embodiment of an ultrasound probe 200 is depicted in FIG. 2, which may be a non-limiting example of the ultrasound probe 108 of FIG. 1. The ultrasound probe 200 has at least one cable 202 that communicatively couples the ultrasound probe 200 to other components of an ultrasound imaging system.

The ultrasound probe 200 also has a housing 204 enclosing components such as elements and actuators for generating and receiving signals. The ultrasound probe 200 may include additional components, such as a metal casing, an acoustic matching layer, an acoustic lens, and a backing material. Each component may have a specific role in moderating an emission and/or reception of ultrasonic waves within the ultrasound probe 200. For example, the backing material may increase an axial resolution of the transmitted ultrasonic signals by dampening excessive vibrations in the ultrasound probe 200 arising from oscillation of an array of elements when a potential is applied. Additionally, an acoustic lens 206 may be disposed in a surface of the housing 204, the acoustic lens 206 configured to focus ultrasonic waves emitted from the elements of the ultrasound probe 200.

During transport of a portable ultrasound imaging system, the ultrasound probe may be subject to degradation if the probe is not securely attached to a main body of the ultrasound imaging system, e.g. the main body 114 of FIG. 1, and comes into contact with other objects. Thus, the main body of the system may be adapted with an accessory holder.

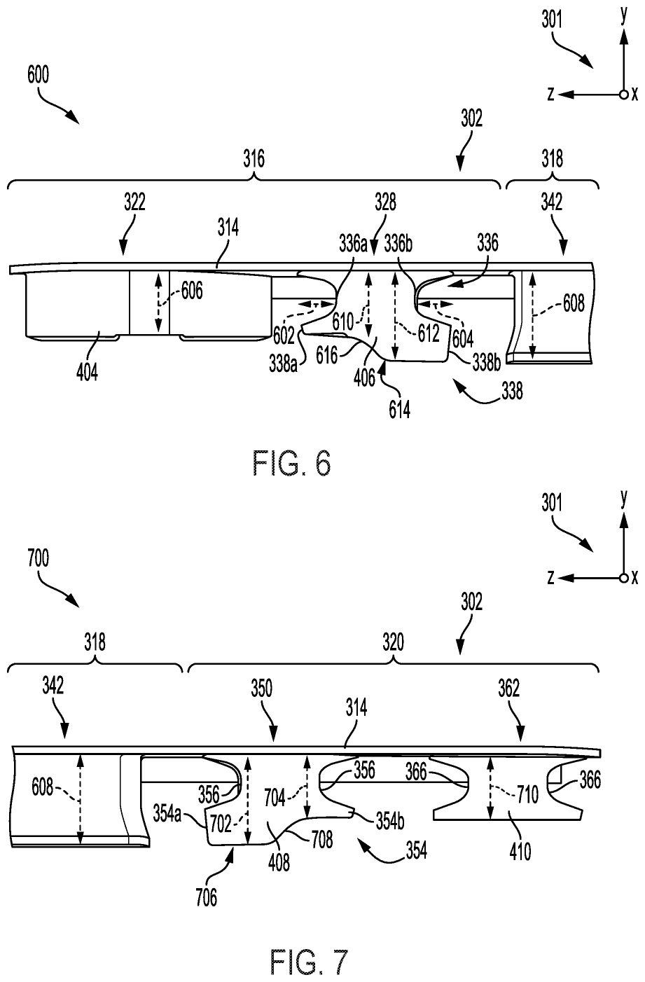

An example of an accessory holder 302 is shown in FIGS. 3-5 in a first perspective view 300, a second perspective view 400 and a front view 500, respectively. In FIGS. 6 and 7, the probe holder 302 is illustrated from a right-side profile view 600 and a left-side profile view 700. As such, FIGS. 3-7 are numbered similarly and described collectively. In one example, the accessory holder 302 may also be a probe holder 302 when implemented in a medical system, such as an ultrasound imaging system. A set of reference axes 301 are provided for comparison between views shown, indicating a y-axis, a z-axis, and an x-axis. The probe holder 302 may be an elongate, plate-like structure with a length 304 along the z-axis (e.g., where the z-axis is a longitudinal axis of the probe holder 302), where the length 304 is greater than a width 306 of the probe holder 302 along the x-axis and a height 308 of the probe holder 302 along the y-axis. The length 304, the width 306 and the height 308 are indicated in FIG. 3. As shown in FIGS. 3-5, the width 306 and the height 308 of the probe holder 302 may not be uniform along the length 304 of the probe holder 302. A front edge 310 of the probe holder 302 may have linear portions, e.g., portions aligned with the z-axis, and a rear edge 312 of the probe holder 302 may be curved relative to the z-axis. The probe holder 302 has a base plate 314 in which a variety of openings may be disposed.

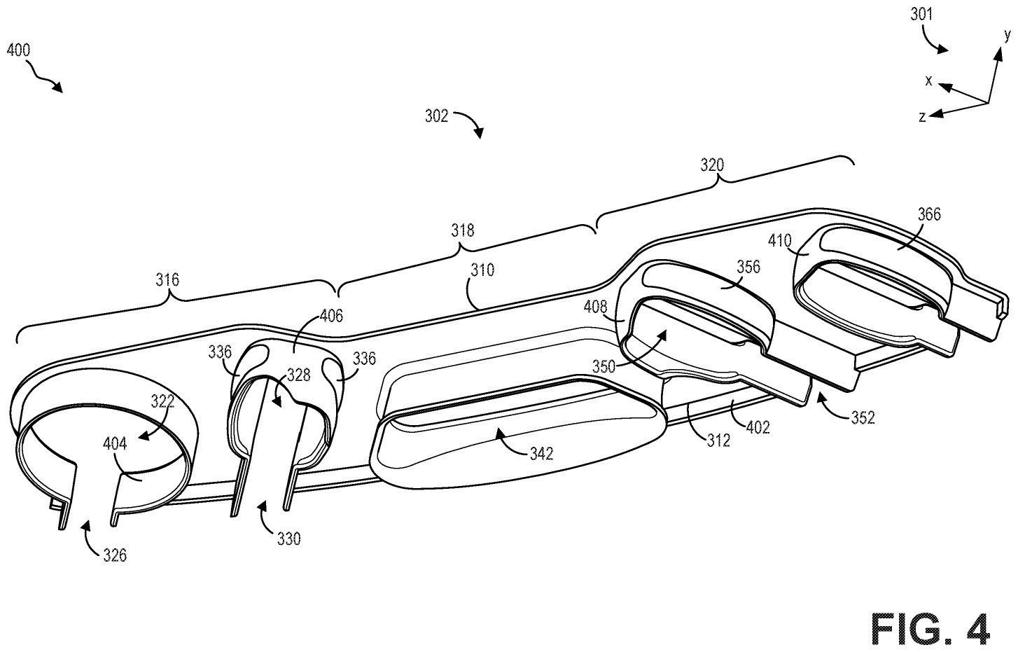

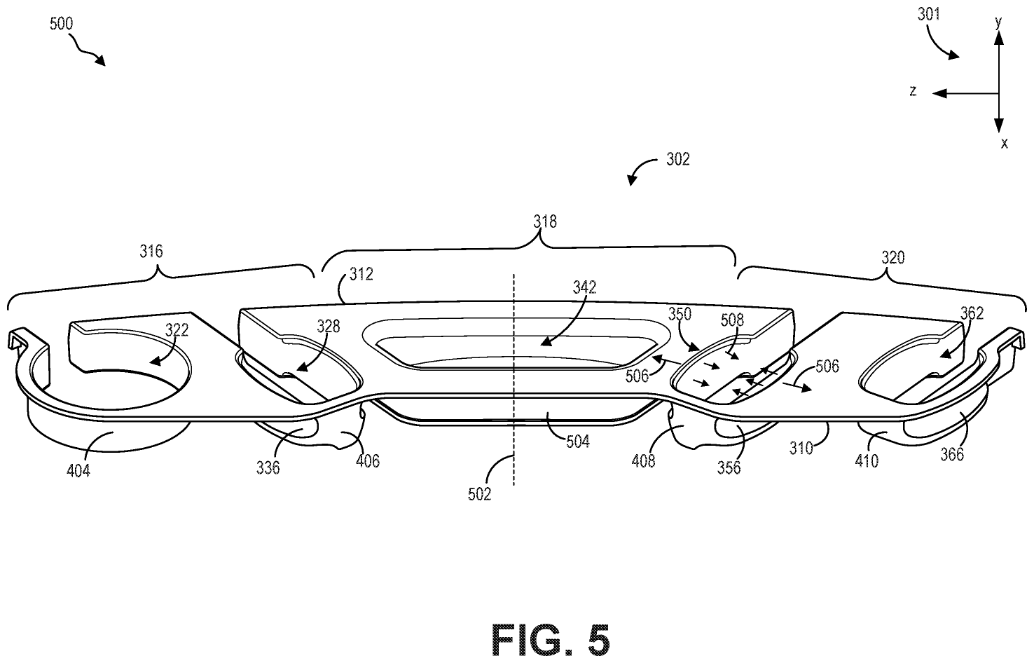

The base plate 314 is flat and co-planar with the x-z plane. The front edge 310 of the probe holder 302 may also be the front edge 310 of the base plate 314. The front edge 310 includes some linear portions and some curved portions, described further below. The rear edge 312, also the rear edge 312 of the base plate 314, may have a uniform radius of curvature, as shown in FIG. 5, and may include a rear lip 402, as shown in FIG. 4, that extends downwards along the y-axis from the rear edge 312 across the length 304 of the probe holder 302.

The base plate 314 has a first, right-side section 316, a second, left-side section 320, and a central section 318 between the first and second sections 316, 320. The first section 316 may include a first opening 322 with a circular geometry and a curved wall 404, as shown in FIG. 3, that circumferentially surrounds the first opening 322 and continues along a first inlet 326 of the first section 316. The curved wall 404 extends downwards in the direction of the y-axis from the base plate 314 around the first opening 322 and the first inlet 326. The first inlet 326 may extend between the first opening 322 and the rear edge 312 of the probe holder 302 so that the first inlet 326 interrupts the rear edge 312. In other words, the rear edge 312 is not continuous along the length 304 of the probe holder 302, as shown in FIG. 5, due to arrangement of a plurality of inlets along the rear edge 312. A diameter 324, as indicated in FIG. 3, of the first opening 322 may be smaller than the width 306 of the probe holder 302 at the first section 316.

The first section 316 of the base plate 314 also has a second opening 328. A second inlet 330 extends between the second opening 328 and the rear edge 312 of the probe holder 302, which may be similar to the first inlet 326. The first inlet 326, the second inlet 330, and the second opening 328 may be similarly angled with respect to the x-axis by an angle α, as shown in FIG. 3. The angle α may be between 0 to +/−180 degrees, in at least one example.

The second opening 328 may have a curved inner surface 406, as shown in FIG. 4, extending downwards from the base plate 314 along the y-axis and surrounding the second opening 328 and continuing along the second inlet 330, similar to the curved wall 404 of the first opening 322. A geometry of the second opening 328 may resemble a rectangle with curved sides, when viewed along the y-axis. A width 332 of the second opening 328, as shown in FIG. 3, may be measured across a distance between sides of the curved inner surface 406 and is wider than a width 334 of the second inlet 330. A set of contact seats 336 may be disposed in the curved inner surface 406 of the second opening 328, along sides 338 of the second opening 328. In other words, the set contact seats 336 are opposing structures, e.g., oppositely positioned, coupled to the curved inner surface 406 of the second opening 328.

The set of contact seats 336 may have surfaces that curve along the y-axis, as shown in FIG. 6, and may be positioned parallel with the second inlet 330, as shown in FIG. 3. The set of contact seats 336 extend along an entire length 340 of the second opening 328. A distance 343 between the set of contact seats 336 may be equal to the width 334 of the second inlet 330. Thus, a positioning of the set of contact seats 336 relative to the second inlet 330 forms a continuous slot of uniform width in the base plate 314.

The inward curvature (e.g., curvature toward the second opening 328) of the set of contact seats 336 is shown in FIG. 6 in a first profile view 600 of the first section 316 of the base plate 314. The set of contact seats 336 curve inwards into the second opening 328 from the sides 338 of the second opening 328. For example, a first contact seat 336a of the set of contact seats 336 curves into the second opening 328, e.g., to the right in FIG. 6, from a first side 338a of the sides 338 of the inner surface 406 of the second opening 328. A second contact seat 338b of the set of contact seats 336 curves in the second opening 328 from a second side 338b of the sides 338, e.g., to the left in FIG. 6. A radius 602 of the first contact seat 336a may be similar to a radius of 604 of the second contact seat 336b but may be different in other examples.

As such, the set of contact seats 336 may be hollow, bubble-like formations that are integrated, e.g., attached continuously and uninterruptedly, into the sides 338 of the second opening 328 and partially close the second opening 328 (e.g., reduce the amount of opening of the second opening 328). The set of contact seats 336 may be continuous with the inner surface 406 of the second opening 328 so that intersecting regions of the set of contact seats 336 with the inner surface 406 are free of crevices or joints. The set of contact seats 336 may be formed from a flexible, durable, soft durometer and chemically resistant material such as silicone and other parts of the probe holder 302, such as the base plate 314, the inner surface 406 of the second opening 328, the curved wall 404 of the first opening 322, etc., may be formed from the same material as the set of contact seats 336. In other examples, the other surfaces or parts may be formed from a less flexible, more rigid material, such as plastic, a resin, a hard durometer rubber, etc. For example, the other parts may be a more rigid material than the set of contact seats 336 if the probe holder 302 is fabricated in a two shot mold. An elasticity of the set of contact seats 336 may be leveraged to maintain a position of a medical device, such as an ultrasound probe, within the second opening 328. Details of a coupling of the medical device to the probe holder 302 are described further below.

By coupling the probe holder 302 to a medical system, such as an ultrasound imaging system, accessory devices such as probes may be secured to the medical system. A probe may be inserted into an opening in the probe holder, the opening including contact seats, e.g., the set of contact seats 336 of FIGS. 3-6. The contact seats may be compressible and bend away from the probe yet exert an amount of pressure on the probe that retains the probe within the opening. An amount of compressive force applied on the probe may be enough to circumvent dislodgement of the ultrasound without causing deformation of the housing.

The probe may be easily engaged with and removed from the probe holder. For example, an operator may pull the probe out of the opening with one hand and similarly replace the probe in the opening with one hand, thereby enabling rapid manipulation of the probe. The contact seats may be formed of a chemically resistant material and coupled continuously to an inner surface of the opening so that the probe holder is formed of smooth, rounded, and continuous surface. As a result, the probe holder may be efficiently sterilized with cleaning chemicals without degrading the material of the probe holder. In one example, the probe holder may be additively manufactured (e.g., 3D printed). Further details of the probe holder are described below, with reference to FIGS. 3-7.

The first profile view 600 of FIG. 6 also shows variations in the height, e.g., the height 308 shown in FIG. 3, of the probe holder 302. For example a height 606 of the curved wall 404 of the first opening 322 may be smaller than a height 608 of the probe holder 302 at the central section 318. A height of the inner surface 406 of the second opening 328 may be non-uniform. The inner surface 406 of the second opening 328 may form a shallow inner cup that extends along a portion of a height of a medical device (not shown in FIG. 6) inserted into the second opening 328. For example, a height 610 of the second opening 328 at the first side 338a may be similar to the height 606 of the curved wall 404 of the first opening 322 and a height 612 of the second opening 328 at the second side 338b may be similar to the height 608 of the central section 318 of the probe holder 302. A bottom edge 614 of the inner surface 406 of the second opening 328 may have a curved portion 616 that transitions between the height 610 of the first side 338a and the height 612 of the second side 338b.

The width 306 of the probe holder 302 at the first section 316 may be wider than at the central section 318 of the probe holder 302, as shown in FIG. 3. The front edge 310 of the base plate 314 has a first sloped portion 341 to transition between the wider first section 316 and the narrower central section 318. The central section 318 has a third opening 342 that does not include an inlet. Thus, the front edge 310 and the rear edge 312 of the base plate 314 are continuous throughout the central section 318. The third opening 342 may be centered within the central section 318 and may have a trapezoidal shape with curved corners when viewed along the y-axis.

The third opening 342 may have a plane of symmetry along the x-y plane as indicated in FIG. 5 by dashed line 502. The third opening 342 has an inner wall 504 surrounding the third opening 342 and protruding downwards from the base plate 314 along the y-axis. The height 608 of the central section 318, as shown in FIG. 6, is a height of the inner wall 504 of the third opening 342. The third opening 342 may be formed from a similar material as the base plate 314 or a similar material as the set of contact seats 336 of the second opening 328 and configured to provide a handhold for the probe holder 302. For example, a width 344 and a length 346 of the third opening 342, as shown in FIG. 3, may be adapted to allow insertion of at least one hand of an operator into the third opening 342 (e.g., for gripping and/or carrying the ultrasound imaging system via the probe holder 302 while the probe holder 302 is coupled to the ultrasound imaging system).

The second section 320 of the base plate 314 is arranged at an opposite of the central section 318 from the first section 316. The second section 320 may have a generally similar shape to the first section 316 as reflected across the y-x plane and similar dimensions, e.g., fraction of overall length 304 of the probe holder 302, but may include one or more openings having a different shape relative to at least one opening of the first section 316, as described below. The width 306 of the base plate 314 at the second section 320 may also be similar to the width 306 of the base plate 314 at the first section 316, which is wider than at the central section 318. The front edge 310 has a second sloped portion 348, as shown in FIG. 3, to accommodate the change in width between the central section 318 and the second section 320.

The second section 320 has a fourth opening 350, with a fourth inlet 352 extending between the rear edge 312 of the base plate 314 and the fourth opening 350. The fourth opening 350 and the fourth inlet 352 may be canted relative to the x-axis by an angle β The angle β may be opposite of the angle α and may be between 0 to +/−180 degrees. In some examples, the fourth opening 350 may have a similar shape as the second opening 328 and the fourth inlet 352 may have a similar width as the width 334 of the second inlet 330. In other examples, the shapes of the openings may vary to accommodate a broad range of probes or other handheld devices.

The fourth opening 350 also has an inner surface 408 that extends down from the base plate 314 along the y-axis, surrounds the fourth opening 350 and continues along the fourth inlet 352. As shown in FIG. 7 in a second profile view 700 of the probe holder 302, a front view of the inner surface 408 of the fourth opening 350 may be a mirror image of a front view of the inner surface 406 of the second opening 328 of the base plate 314, reflected across the y-x plane. A height of the inner surface 408 of the fourth opening 350 is non-uniform, having a height 702 at a first side 354a of sides 354 of the inner surface 408 that is similar to the height 608 of the third opening 342 of the base plate 314 and a height 704 at a second side 354b of the sides 354 of the inner surface 408 that is similar to the height 606 of the first opening 322 (as shown in FIG. 6). A bottom edge 706 of the inner surface 408 of the fourth opening 350 has a sloped portion 708 between the first side 354a and the second side 354b of the inner surface 408.

As shown in FIGS. 3-5, the fourth opening 350 is similarly configured as the second opening 328 with a set of contact seats 356 which have surfaces that are linear along the y-x plane and curved along the y-z plane. A material of the set of contact seats 356 may be more flexible than a material of the inner surface 408 of the fourth opening 350. The set of contact seats extend along an entire length 351 of the fourth opening 350, as shown in FIG. 3, and are seamlessly integrated or molded into the inner surface 408 of the fourth opening 350. The set of contact seats 356 curve inwards along the y-axis, into the fourth opening 350, as illustrated in FIG. 7. Along the x-z plane, the set of contact seats 356 extend linearly so that a distance 358 between the set of contact seats 356 of the fourth opening 350 is uniform along the length 351 of the fourth opening 350 and equal to a width 360 of the fourth inlet 352, as shown in FIG. 3. Together, the fourth inlet 352 and a space between the set of contact seats 356 of the fourth opening 350 form a slot in the base plate 314 with a uniform width.

The second section 320 of the base plate 314 further includes a fifth opening 362. A fifth inlet 364 extends between the rear edge 312 of the base plate 314 and the fifth opening 362. The fifth opening 362 and the fifth inlet 364 are angled relative to the x-axis by the angle (3, similar to the fourth opening 350 and fourth inlet 352. The fifth opening 362 has an inner surface 410, as shown in FIG. 4, which extends down from the base plate 314 along the y-axis, surrounding the fifth opening 362 and continuing along the fifth inlet 364. Similar to the fourth opening 350 and the second opening 328, the inner surface 366 includes a set of contact seats 366, continuous with the inner surface 410 and extending along an entire length 368 of the fifth opening 362, as shown in FIG. 3. The set of contact seats 366 are similarly shaped as the set of contact seats 356 of the fourth opening 350 as well as the set of contact seats 336 of the second opening 328.

While an overall shape of the fifth opening 362 may resemble the overall shapes of the second opening 328 and the fourth opening 350, dimensions of the fifth opening 362 may differ from the second opening 328 and the fourth opening 350. For example, as shown in FIG. 3, the length 368 of the fifth opening 362 may be greater than the length 351 of the fourth opening 350. A width 370 of the fifth opening 362, as well as a distance 372 between the set of contact seats 366 of the fifth opening 362, may be larger than a width 374 of the fourth opening 350 and the distance 358 between the set of contact seats 356 of the fourth opening 350, respectively.

Furthermore, as shown in FIG. 3, the fifth inlet 364 may have a width 376 that is greater than the width 360 of the fourth inlet 352 and a length 378 that is shorter than a length 380 of the fourth inlet 352. A height 710 of the inner surface 410 of the fifth opening 362, as indicated in FIG. 7, may be uniform throughout the fifth opening 362 and may be similar to the height 704 of the fourth opening 350 at the second side 354b of the fourth opening 350 as well as similar to the height 606 of the curved wall 404 of the first opening 322 (as shown in FIG. 6). The second profile view 700 of FIG. 7 also depicts an inwards curving of the set of contact seats 366 of the fifth opening 362, into the fifth opening 362.

It will be appreciated that the probe holder 302 is a non-limiting example of a holder adapted to support hand-held medical devices. A number of openings, sizes and shaped of the openings, and spacing of the openings relative to one another may be varied without departing from the scope of the present disclosure.

The sets of contact seats of each of the second, fourth, and fifth openings 328, 350, 362, may all be formed from a same flexible material with an elasticity that enables the material to readily deform when a force is applied to the material and return to its original shape when the force is removed. For example, the material of the contact seats may have a Shore A durometer hardness of 65-70. Each of the openings of the probe holder 302 are configured to receive an object, e.g., a medical device, that directly contacts the contact seats of each of the openings. Each opening may retain an object with a width greater than a distance between the set of contact seats disposed in the opening but narrower than the width of the opening. As such, the fifth opening 362 may be used to support an object that is wider than the widths of the second opening 328 and the fourth opening 350. For example, a medical device may be inserted into each of the first, second, fourth, and fifth openings of the probe holder 302 as shown in FIG. 8 in a perspective view 800 of an ultrasound imaging unit 802.

The ultrasound imaging unit 802 may be a portable unit which may be handheld or carried with a strap coupled to a housing 804 of the ultrasound imaging unit 802. In one example, the ultrasound imaging unit 802 may be the main body 114 of FIG. 1, configured to be removable from the cart 104 and brought to a treatment site to provide point-of-care use. The ultrasound imaging unit 802 also has a display 806 arranged at a front side of the ultrasound imaging unit 802. The housing 804 may frame the display 806 at the front side of the ultrasound imaging unit 802 and extend upwards, above the display 806 to form a top shelf 808. A plane of the top shelf 808 may be angled relative to the x-z plane and the y-z plane. The top shelf 808 may have an edge 810 shaped to match a geometry of the front edge 310 of the base plate 314 of the probe holder 302.

The probe holder 302 may couple continuously, e.g., without any breaks or gaps, to the top shelf 808. In other words, the base plate 314 of the probe holder 302 is in edge-sharing contact along the edge 810 of the top shelf 808 of the ultrasound imaging unit 802 along the entire length 304 of the probe holder 302 (the length 304 shown in FIG. 3). When coupled to the ultrasound imaging unit 802, the probe holder 302 may no longer be co-planar with x-z plane. Instead, the probe holder 302 may be coupled to the top shelf 808 so that a plane of the probe holder 302 is co-planar with the plane of the top shelf 808. In one example, the base plate 314 of the probe holder 302 may be attached to the top shelf 808 of the ultrasound imaging unit 802 by an adhesive. In another example, the base plate 314 may be coupled to the top shelf 808 by clips, fittings that lock into place when the base plate 314 is pressed against the top shelf 808, or by various other removable or non-removable fastening devices and mechanisms. In yet another example, the base plate 314 may be formed together (e.g., molded together) with the top shelf 808 as a single unit.

A positioning of the probe holder 302 at a top, with respect to the y-axis, of the ultrasound imaging unit 802, allows medical devices, such as ultrasound probes, to be supported and secured in place by the probe holder 302. The medical devices may be readily accessed by an operator and placed out of the way when not in use. It will be appreciated that the ultrasound imaging unit 802 is a non-limiting example of a system to which the probe holder 302 may be coupled. The probe holder 302 may be similarly used for various other systems with removable medical devices, for various other applications including cardiology, radiology, non-portable systems, etc.

As shown in FIG. 8, the first opening 322 of the probe holder 302 may be configured to support a cup 812. The cup 812 may be inserted into the first opening 322 and held in place by a lip 814 of the cup 812. The cup 812 may have a slot 816 that is aligned with the first inlet 326 and has a width similar to a width of the first inlet 326.

A first medical device 818 may be inserted into the cup 812 and supported by the cup 812, and a cable of the first medical device 818 may be fed through the first inlet 326 and slot 816 of the cup 812 such that the cable of the first medical device 818 hangs downwards behind the ultrasound imaging unit 802. The second opening 328 of the probe holder 302 is depicted in FIG. 8, without a medical device inserted into the second opening 328. However, the second opening 328 may be configured to retain a medical device in a similar manner as described below for the fourth opening 350 and the fifth opening 362 of the probe holder 302.

A second medical device 820, which may be an ultrasound probe, is shown inserted into the fourth opening 350 of the probe holder 302. In order to engage the second medical device 820 with the fourth opening 350, the second medical device 820 may first be positioned above the fourth opening 350, with a cable of the second medical device 820 passing through the fourth inlet 352. The second medical device 820 may then be lowered into the fourth opening 350. A width 822 of the second medical device 820, at a handle 824 of the second medical device 820, may be wider than the distance 358 (shown in FIG. 3) between the set of contact seats 356 of the fourth opening 350, but narrower than the width 374 (also shown in FIG. 3) of the fourth opening 350. Furthermore, the width 822 of the second medical device 820 is wider than a width 360 (as shown in FIG. 3) of the fourth inlet 352 thereby inhibiting sliding of the second medical device 820 through the fourth inlet 352.

Lowering the second medical device 820 into the fourth opening 350 may thus include pressing the second medical device 820 against a resistance provided by the set of contact seats 356 of the fourth opening 350. A hardness of the set of contact seats 356, e.g., a desired amount of flexibility of a material of the set of contact seats 356, allows the set of contact seats 356 to be compressed and deform in response to engagement with the less flexible housing 804 of the ultrasound imaging unit 802. Deformation of the set of contact seats 356 includes flexing of the set of contact seats 356 outwards in a first direction, away from a central region of the fourth opening 350, as indicated by arrows 506 shown in FIG. 5. The material of the set of contact seats 356 provides resistance as the material is forced outwards by insertion of the second medical device 820, exerting an opposing, inwards force (e.g., restoring force) in a second direction, e.g., towards the central region of the fourth opening 350, as indicated by arrows 508 in FIG. 5. The flexing of the sets of contact seats of the probe holder 302 is described further below with reference to FIGS. 9-10.

In some examples, the inwards facing surfaces, e.g., surfaces that directly contact a medical device of a set of contact seats (e.g., contact seats 356) may be textured or structurally modified to adjust the contact between the set of contact seats and the medical device. In one example, the surfaces of the set of contact seats may include ridges or some other small protrusions so that the surfaces are not smooth and friction between the set of contact seats and the medical device is increased. In other examples, the surfaces may be adapted with one or more indentations, or dimples, to adjust a compressibility of the set of contact seats. An example of a set of contacts seats with dimples is depicted in FIG. 11.

As shown in FIG. 11 in a partial view 1100 of a probe holder 1102, an opening 1104 similarly includes a set of contact seats 1106. The opening 1104 and the set of contact seats 1106 are symmetric across a central axis 1108 of the opening 1104. Each contact seat of the set of contact seats 1106, in some examples, may be adapted with a dimple 1110, arranged in a central region of each contact seat. The dimple 1110 may be an indentation in each contact seat, configured with an opposite curvature than the contact seat along the y-axis. For example, the dimple 1110 curves outwards, away from the central axis 1108 both along the y-axis and along the x-axis. The dimple 1110 may have a radius of curvature that is smaller than a radius of curvature of the set of contact seats 1106. In other examples, the contact seats may have a plurality of dimples or other resistance structures. These dimples may be similarly sized or may have variable sizes.

Implementing the dimple 1110 in each contact seat of the set of contact seats 1106 may reduce a resistance of the set of contact seats towards flexing outwards, away from the central axis 1108, when a medical device is inserted into the opening 1104. A hardness, e.g. a durometer value, of a material of the set of contact seats 1106 may be adjusted according to presence of the dimple 1110. For example, a less flexible material may be used to form the set of contact seats 1106 when the dimple 1110 is included than when the dimple 1110 is not included. As noted above, in other examples, more than one dimple 1110 may be disposed in each of the set of contact seats 1106 allowing an even less flexible material to be used for the set of contact seats 1106.

Returning to FIG. 8, the inwards force is exerted on the second medical device 820 in radial directions. In other words, the material of the set of contact seats 356 presses against the handle 824 of the second medical device 820. Friction generated between the set of contact seats 356 and the handle 824 of the second medical device 820 by a compressive force exerted on the handle 824 by the material of the set of contact seats 356 maintains a position of the second medical device 820 within the fourth opening 350 of the probe holder 302. In other words, the second medical device 820 is gripped by the set of contact seats 356 which inhibits sliding, rocking, and bouncing of the second medical device 820 within the fourth opening 350 when the ultrasound imaging unit 802 is transported while an operator is running, for example.

Flexing of a set of contact seats is depicted in greater detail in FIGS. 9-10. Therein, an opening 902 is illustrated which may be any of the second, third or fourth openings 328, 350 or 362 of the probe holder 302. The opening 902 may also represent a shape of an inner cup of the opening 902. A central axis 901 extends through the opening 902, parallel with the x-axis, and the opening 902 may be symmetric across the central axis 901 as well as a set of contact seats 904. The opening 902 is equipped with the set of contact seats 904, similar to the sets of contact seats shown in FIGS. 3-8 and shown in a first configuration 900 in FIG. 9 and a second configuration 1000 in FIG. 10. The set of contact seats 904 have inner surfaces 906 arranged parallel with the x-axis in the first configuration 900 of FIG. 9 and curved inwards, e.g., are concave, towards the central axis 901 along the y-axis. This first configuration 900 may represent when the set of contact seats 904 are not engaged with a medical device.

In the second configuration 1000 of FIG. 10, a medical device 1002 is inserted into the opening 902, aligned parallel with the y-axis. A first width 1008 of the medical device 1002, measured along the z-axis, is greater than a space 1010 between the set of the contact seats but smaller than a width 1012 of the inner cup of the opening 902. A second width 1014 of the medical device 1002, measured along the x-axis, is smaller than a length 1016, also defined along the x-axis, of the set of contact seats 904.

As the medical device 1002 is pushed down, along the y-axis, into the opening 902, between the set of contact seats 904, contact between the medical device 1002 compresses the set of contact seats 904 and pushes against the set of contact seats 904 so that at least a portion 1004 of the inner surfaces 906 of each contact seat of the set of contact seats 904 bends away from the central axis 901. In other words, a distance between the set of contact seats 904, e.g., the space 1010, increases to be similar to the width 1008 of the medical device 1002 at a central region of the portion 1004 of the inner surfaces 906 of the set of contact seats 904. As such, the portion 1004 of the inner surfaces 906 of each contact seat becomes convex, e.g., curving away from the central axis 901, along the x-axis and may be linear along the y-axis. However, a change in shape of the portion 1004 of the inner surfaces 906 of each contact seat may depend on a geometry of the medical device 1002 and conform to the geometry of the medical device 1002. For example, if the medical device 1002 has straight sides, the portion 1004 of the inner surfaces 906 of each contact seat may be similarly shaped. The portion 1004 of the inner surfaces 906 of each contact seat that bends away from the central axis 901 is in direct contact with the medical device 1002 and may mold to an outer shape of the medical device 1002. Portions 1006 of the inner surfaces 906 of each contact seat that are not in contact with the medical device 1002 may remain parallel with the x-axis and concave along the y-axis.

Returning to FIG. 8, the extension of the set of contact seats 356 along the entire length 351 of the fourth opening 350 allows contact between the set of contact seats 356 and the second medical device 820 to be maximized. By maximizing contact, rocking of the second medical device 820 within the fourth opening 350 is circumvented, where a direction of rocking is indicated by arrow 826 in FIG. 8. Rocking is further mitigated by resistance provided by the portions of the set of contact seats 356 that are not in contact with the second medical device 820, e.g., the portions 1006 of FIG. 10.

The second medical device 820 may be removed from the fourth opening 350 by pulling the second medical device 820 upwards, along the y-axis and away from the set of contact seats 356 of the fourth opening 350. The hardness/flexibility of the set of contact seat 356 may enable the second medical device 820 to be easily removed with one hand of the operator. Upon disengagement of the second medical device 820, the hardness/flexibility of the set of contact seats 356 of the fourth opening 350 may return to first configuration 900. The cable coupled to the second medical device 820 may be passed through the fourth inlet 352 to fully disengage the second medical device 820 from the probe holder 302. The third inlet allows the cable of the second medical device 820 to hang downwards from the second medical device 820 behind the ultrasound imaging unit 802 when the second medical device 820 is retained in the fourth opening 350, thereby positioning the cable away from the operator. A likelihood of entanglement with the cable or inadvertent pulling of the cable by the operator or a subject is thus reduced. Furthermore, bending, wrapping, or other manipulation of the cable is not demanded.

The second opening 328 of the probe holder 302 may be configured to receive a medical device similar in size and shape to the second medical device 820. The fifth opening 362, however, may be configured to engage with a third medical device 828 with larger dimensions than the second medical device 820 due to the larger width and length of the fifth opening 362 relative to the fourth opening 350, as well as the greater distance between the set of contact seats 366 of the fifth opening 362 compared to the distance between the set of contact seats 356 of the fourth opening 350, as shown in FIG. 3. As well, the cup 812 inserted into the first opening 322 of the probe holder 302 may be adapted to receive small medical devices that are too small to be supported by the second opening 328, the fourth opening 350, or the fifth opening 362.

By providing the probe holder 302 with openings of different shapes and sizes, medical devices of various geometries and dimensions may be supported by the probe holder 302. Adapting each opening with an inlet mitigates tangling of device cables with one another or with the operator's hands, maintaining the cables at a rear side of the ultrasound imaging system. It will be appreciated that the example of the probe holder shown in FIGS. 3-8 is a non-limiting example and variations in a configuration of the probe holder have been contemplated. As such, other examples of the probe holder may include different ordering of the openings shown in FIGS. 3-8 along the length of the probe holder, different shapes of the openings, different quantities of the openings, etc. Furthermore, the probe holder may be adapted to couple to other systems besides the ultrasound imaging system.

In this way, accessories, such as probes, may be secured to a unit, such as an ultrasound imaging unit, thus decreasing a likelihood of the accessories becoming separated from the unit during transport. The unit may be equipped with an accessory holder configured with openings adapted to receive the accessories. The openings may include inlets, through which cables of the accessories may be guided, and contact seats integrated seamlessly and continuously into the openings. When in a first configuration and not engaged with an accessory, the contact seats may have inner surfaces that are aligned parallel relative to a horizontal axis and curved relative to a vertical axis. The contact seats may be formed of a material with a durometer value that allows the contact seats to flex outwards into a second configuration when the probe is pushed into a space between the contact seats of each opening while providing enough friction between the contact seats and the accessory to grip the accessory and maintain a position of the accessory. Adapting the openings with the contact seats allows each opening to accommodate a variety of accessory sizes and shapes. The accessories may be single-handedly coupled to and decoupled from the contact seats of the openings and the seamless integration of the contact seats into the openings allows the accessory holder to be efficiently cleaned.

The technical effect of adapting a unit with the accessory holder configured to retain one or more accessories of the unit via flexible contact seats integrated into openings of the accessory holder is that securing of the one or more accessories to the unit is enabled, particularly during transport of the unit, while allowing the accessory holder to be easily sanitized.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An accessory device holder, comprising:
a base plate with a front edge and a rear edge; and
an inlet extending from the rear edge of the base plate to an opening in the base plate, wherein the opening includes oppositely arranged, rebounding contact seats that partially close the opening, where the contact seats are configured to bend away from an accessory inserted into the opening while exerting a pressure on the accessory,
wherein surfaces of the contact sets are one of textured and structurally modified to increase friction between the contact seats and the accessory.

2. The accessory holder of claim 1, wherein the contact seats are hollow, bubble-like structures with surfaces that are linear along a horizontal axis of the holder and curve inwards towards a central axis of the opening along a vertical axis of the holder.

3. The accessory holder of claim 1, wherein the contact seats are coupled to an inner cup of the opening and wherein intersecting regions of the contact seats with the inner cup are continuous and uninterrupted.

4. The accessory holder of claim 3, wherein the contact seats are formed from a more compressible and elastic material than the inner cup of the opening and the base plate.

5. The accessory holder of claim 4, wherein a first width of the accessory is greater than a space between the contact seats and narrower than a width of the opening and wherein a second width of the accessory is less than a length of the contact seats, where the first width, the second width, and the width of the opening are perpendicular to a central axis of the opening and the length of the contact seats is parallel with the central axis.

6. The accessory holder of claim 5, wherein the length of the contact seats extends along an entire length of the opening and wherein the space between the contact seats is similar to a width of the inlet.

7. The accessory holder of claim 5, wherein portions of the contact seats in direct contact with the accessory conform to an outer shape of the accessory and exert a compressive force on the accessory.

8. The accessory holder of claim 7, wherein when the accessory is inserted into the opening, the contact seats flex outwards, away from the central axis and wherein the space between the contact seats are widened to the width of the accessory at the portions of the contact seats in direct contact with the accessory.

9. The accessory holder of claim 1, wherein the accessory holder has a plurality of inlets and a plurality of openings and wherein each inlet of the plurality of inlets is coupled to one opening of the plurality of openings.

10. A holder for a system, comprising:
a plurality of openings in a plate, each opening having a set of curved elastic contact seats arranged on opposite sides and curving into a space of each opening, the contact seats configured to flex outwards and away from a central axis of each opening to increase a distance between the set of contact seats when an object is inserted and to decrease the distance between the set of contact seats when the object is removed, wherein surfaces of the contact seats are at least one of textured and structurally modified to increase friction between the surfaces and the object.

11. The holder of claim 10, wherein each opening and the set of contact seats positioned in each opening are symmetric about the central axis of each opening.

12. The holder of claim 10, wherein regions of the set of contact seats in contact with the object are pushed outwards, away from the central axis, by the object and wherein a hardness of the set of contact seats exerts a compressive force on the object, the compressive force directed inwards, towards the central axis.

13. The holder of claim 10, further comprising a first configuration of the set of contact seats when the object is not inserted, wherein the set of contact seats are linearly aligned along the central axis of each opening and concave along a vertical plane of the holder, the vertical plane perpendicular to the central axis.

14. The holder of claim 13, further comprising a second configuration of the set of contact seats when the object is inserted, wherein the set of contact seats conform to a shape of the object along regions of the set of contact seats in contact with the object.

15. The holder of claim 14, wherein regions of the set of contact seats not in contact with the object remain in the first configuration when the object is inserted.

16. The holder of claim 10, wherein the set of contact seats includes concave indentations arranged in a central region of each of the set of contact seats.

17. A probe holder for an ultrasound system, comprising:
a base configured to be coupled to a portable ultrasound unit and having a plurality of openings;
a set of hollow, conformable contact seats arranged in each of the plurality of openings with at least one of texturing and structural modification applied to surfaces of the set of contact seats; and
a plurality of slots extending from an edge of the base to each of the plurality of openings and having narrower widths than the plurality of the openings.

18. The probe holder of claim 17, wherein a probe of the ultrasound system is inserted into at least one opening of the plurality of openings, the probe having a cable coupling the probe to the ultrasound system and wherein the set of contact seats in the opening are configured to flex and press against the probe and a slot of the plurality of slots, the slot coupled to the opening, is configured to receive the cable.

19. The probe holder of claim 18, wherein a diameter of the probe is greater than the widths of the plurality of slots and smaller than the widths of the plurality of openings.

20. The probe holder of claim 17, wherein the probe holder is fabricated by additive manufacturing to be a single, continuous unit with a central opening configured to receive an operator's hand.
